(19) United States
(12) Patent Application Publication
(10) Pub. No.: US 2004/0097944 A1
(43) Pub. Date: May 20, 2004

(54) Fixation device and method for treating contractures and other orthopedic indications

(76) Inventors: L. Andrew Koman, Winston-Salem, NC (US); Gregory A. Mohler, Pfafftown, NC (US); Graziano Rossi, Verona c.a.p. (IT); Luigi Rossi, Peschiera del Garda (IT); Richard M. Bryant, Clemmons, NC (US)

Correspondence Address: BAKER BOTTS L.L.P., 2001 ROSS AVENUE, SUITE 600, DALLAS, TX, 75201-2980, US

(21) Appl. No.: 10/629,192

(22) Filed: Jul. 29, 2003

Related U.S. Application Data
(60) Provisional application No. 60/399,878, filed on Jul. 30, 2002.

(51) Int. Cl. A61B 17/56; A61F 2/30

(52) U.S. Cl. 606/72

(57) ABSTRACT

The present invention includes a fixation device for treatment of orthopedic indications. The device preferably includes a first portion and a second portion which may be incrementally and independently articulated relative to each other to promote healing of an orthopedic indication. One or more worm gear assemblies may be used to incrementally adjust articulation of the first portion and the second portion relative to each other.

### RELATED APPLICATIONS

This application claims the benefit of previously filed provisional application entitled “External Fixator and Method for Treating Contractures and Other Orthopedic Indications” serial number 60/399,878 filed Jul. 30, 2002.

### TECHNICAL FIELD

The present invention is generally related to orthopedic devices and more particularly to fixation devices associated with treating joint and soft tissue contractures, bone lengthening and various types of bone fractures.

### BACKGROUND OF THE INVENTION

Various medical conditions exist which may be characterized as or may lead to joint deformities after ligament, tendon or other soft tissue contractures. Such conditions include, but are not limited to, radial club hand, arthrogrypotic knee, clubfoot, severe burns, or frostbite and other trauma to soft tissue. The range of motion in affected joints may be significantly diminished in individuals suffering one or more of the above conditions. Historically, various methods including splinting, casting and surgical release have been used to treat such conditions. Recent advances in treatment have been made through use of devices that mechanically stretch affected joints and/or soft tissue over a period of time. U.S. Pat. No. 6,063,0872 to Agee et al. describes one method to increase the range of motion of finger joints which have been injured by contracture. The method includes attaching external fixation to bones both proximal and distal to the affected joint. Torque may then be applied to the external fixator to dynamically stretch soft tissue associated with the joint.

There are several different types of fixators currently being used that have multiple degrees of freedom and thus may be applied in a manner that generally conforms with physiological geometry of an affected joint. Examples of external fixators are shown in U.S. Pat. No. 6,010,501 to Raskin et al. and U.S. Pat. No. 6,171,309 to Huebner. Such devices often rely on a ball and socket assembly to achieve increased range of motion. Such fixation devices may sometimes prohibit a physician from performing necessary adjustments to obtain desired joint correction and at the same time maintain desired torque of the devices. It may also be difficult to mechanically adjust each degree of freedom independently from another degree of freedom. The range of motion provided by such devices may be insufficient for treatment of some types of joint contractures.

External fixation devices which allow pivotal or rotational movement in only a vertical plane are commercially available. External fixation devices which allow pivotal or rotational movement in only a horizontal plane are also commercially available.

### SUMMARY OF THE INVENTION

In accordance with teachings of the present invention, a fixation device and method are provided with an increased number of degrees of freedom and incremental controls for adjusting each degree of freedom to enhance treatment of contractures, bone fractures, bone lengthening and other orthopedic indications. Skeletal joints with complex types of motion such as a wrist or an ankle often require fixation with substantially enhanced degrees of freedom for proper treatment. One aspect of the present invention includes an external fixator satisfactory for use in repairing and/or treating any type of joint and/or soft tissue contracture alternatively, teachings of the present invention may be used with an internal fixator.

Technical benefits of the present invention include a fixation device with independently controlled rotation in both horizontal and vertical planes. The fixation device may also include independently controlled clamp assemblies. For one embodiment, two worm gears assemblies may be provided to allow controlled, incremental rotation or angulation in a generally horizontal plane and at the same time controlled, incremental rotation or angulation in a generally vertical plane. Respective driver screws may also be provided to allow controlled, incremental positioning of associated clamp assemblies. The present invention provides independent, incremental control of each degree of freedom of an associated fixation device.

Fixators incorporating teachings of the present invention may be used to treat a wide variety of contractures and other orthopedic indication and is not limited to a specific type of contracture or orthopedic indication. Such fixators may be adopted for use with both internal and external fixation techniques and methods.

For some applications an external fixation device incorporating teachings of the present invention may be particularly adapted for engagement with relatively small bones associated with the metacarpus. For other applications an external fixation device incorporating teachings of the present invention may be adapted for engagement with any other bones of a patient.

### BRIEF DESCRIPTION OF THE DRAWINGS

A more complete understanding of the present invention and advantages thereof may be acquired by referring to the following description taken in conjunction with the accompanying drawings in which like reference numbers indicate like features and wherein:

FIG. 1 is a schematic drawing showing an isometric view of a fixation device attached to the wrist of a patient in accordance with teachings of the present invention;

FIG. 2 is a schematic drawing showing an isometric view of the fixation device of FIG. 1 in an aligned or non-rotated position;

FIG. 3 is a schematic drawing showing the fixation device of FIG. 2 in an articulated or rotated position;

FIG. 4 is a schematic drawing with portions broken away taken along lines 4-4 of FIG. 3; and

FIG. 5 is a schematic drawing showing an isometric, exploded view of various components associated with the fixation device of FIG. 2.

### DETAILED DESCRIPTION OF THE INVENTION

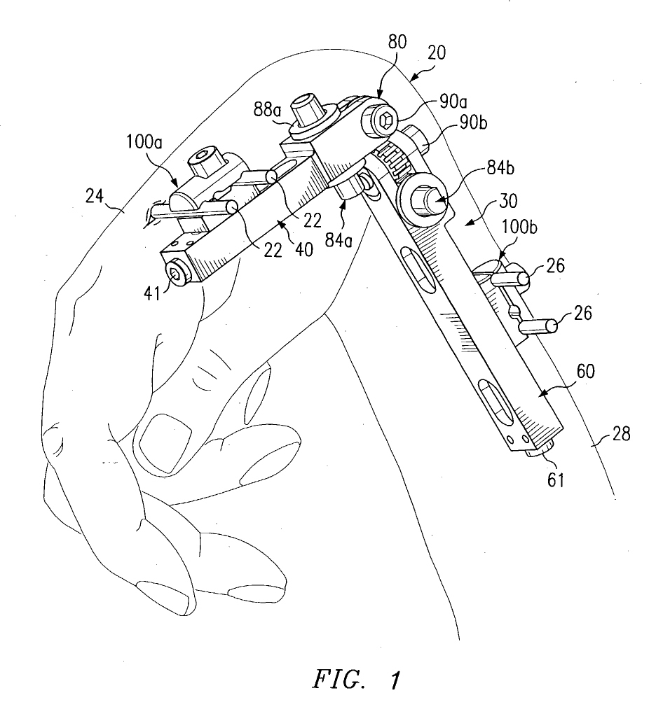

The present invention and its advantages are best understood by reference to FIGS. 1-5 of the drawings, like numerals being used for like and corresponding parts.

The terms “contracture” and “contractures” are used in this application to mean any type of joint or soft tissue deformity or distortion or any other lack of motion in a joint. Contractures may be caused by shortening of an associated muscle, ligament and/or tendon. Contractures may also result from scar tissue or damage to a joint capsule. Many contractures result from a combination of muscle changes and joint capsule changes. Treatment of contractures often requires extensive rehabilitation.

Fixation equipment and methods incorporating teachings of the present invention may be satisfactorily used to treat various orthopedic indications and contractures including, but not limited to, congenital deformation such as radial club hand, arthrogrypotic knee, clubfoot equinovarus/equinovalgus, thermal injuries such as burn and frostbite, soft tissue damage from stroke, trauma or tumor, spasticity (muscular hypertonicity with increased tendon reflexes), thumb or finger contractures, surgical release, comminuted distal metacarpal fracture and open reduction/internal fixation. The term “orthopedic indications” is used in this application to include the previously noted indications and any similar or related medical conditions.

FIG. 1 is a schematic drawing showing one example of an external fixation device incorporating teachings of the present invention attached to a patient's arm and hand for use in treating a wrist contracture. For this example of treating a contracture at wrist 20, a first pair of bone pins or screws 22 may be securely installed with the second metacarpal (not expressly shown) of hand 24. A second set of bone pins 26 may be securely engaged with the radius (not expressly shown) of arm 28. Conventional surgical procedures may be used to install bone pins 22 and 26 at desired locations relative to wrist 20. The number of pins and location of the pins will often vary depending upon characteristics of the associated contracture and physiological condition of the patient's hand, arm and associated soft tissue. Also, any type of bone pin or bone screw may be satisfactorily used with a fixation device incorporating teachings of the present invention. The present invention is not limited to use with pins 22 and 26 as shown in FIG. 1. The present invention is not limited to external fixation devices as shown in FIGS. 1-5.

External fixation device 30 incorporating teachings of the present invention may sometimes be referred to as an external fixator. External fixator 30 may be used to treat a wide variety of contractures in skeletal joints, either congenital or acquired. However, fixation equipment and methods incorporating teachings of the present invention may be used in other orthopedic applications including, but not limited to fractures and bone lengthening. External fixation device 30 may be satisfactorily used to treat a wide variety of orthopedic indications. A fixation device incorporating teachings of the present invention may be formed from a wide variety of materials. For some applications external fixation device 30 may be formed from aluminum and/or stainless steel or other metal alloys satisfactory for use in treating orthopedic indications. For other applications various components and parts associated with external fixation device 30 may be formed from high strength composite materials and/or cermets.

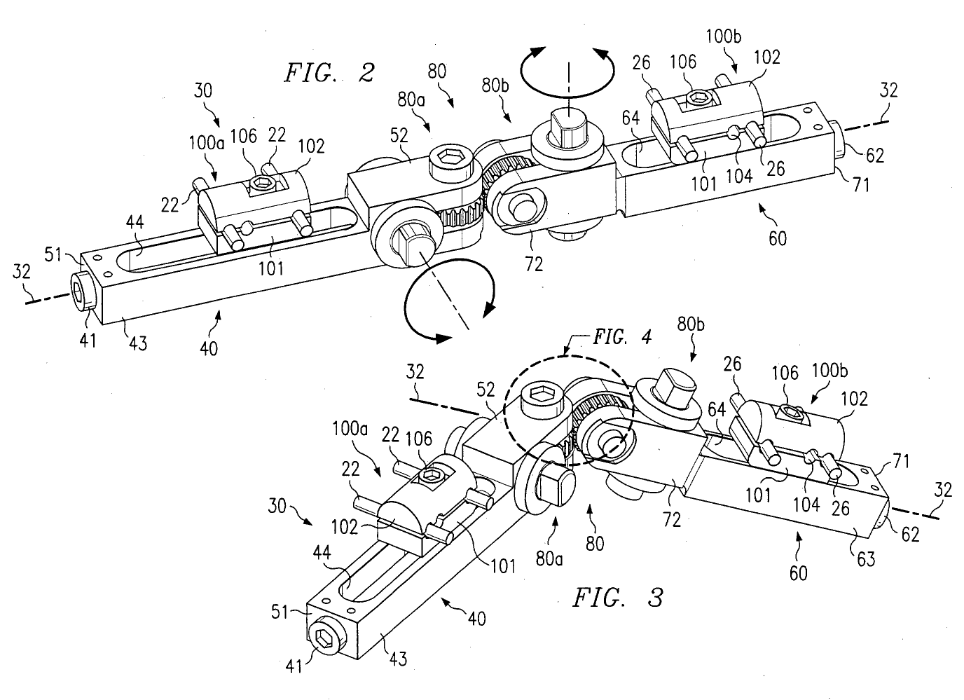

For the embodiment of the present invention as shown in FIGS. 1-5, external fixation device 30 preferably includes first portion 40, second portion 60 and coupling assembly 80 disposed therebetween. For the example shown in FIG. 1, first portion 40 may be releasably attached with the second metacarpal in hand 24. Second portion 60 may be releasably attached with the radius in arm 28. Coupling assembly 80 may be used to incrementally position first portion 40 and second portion 60 as desired to treat contracture of wrist 20. In FIG. 2, first portion 40, second portion 60 and coupling assembly 80 are shown generally aligned with each other extending along center line or longitudinal axis 32. In FIG. 3, first portion 40 is shown in a rotated or articulated position relative to second portion 60 and center line or longitudinal axis 32. As discussed later in more detail, coupling assembly 80 allows independent, incremental rotation or articulation of first portion 40 and second portion 60 relative to each other.

First portion 40 and second portion 60 preferably include respective clamp assemblies 100a and 100b. For the embodiment of the present invention as shown in FIGS. 1-3 clamp assemblies 100a and 100b are substantially identical. For other applications first portion 40 and second portion 60 may have respective clamp assemblies with different configurations and/or dimensions. Since clamp assemblies 100a and 100b are substantially identical and the relationship between clamp assembly 100a and first portion 40 and clamp assembly 100b and second portion 60 are substantially identical, only clamp assembly 100a will be described in detail.

For the embodiment of the present invention as shown in FIGS. 1-3 clamp assembly 100a preferably includes first jaw 101 and second jaw 102. Jaw 101 may sometimes be referred to as a carriage. Each jaw 101 and 102 preferably includes a plurality of grooves 104 which are sized to receive respective bones pins 22 and 26. For the embodiment of the present invention represented by external fixation device 30, grooves 104 have approximately the same dimensions and configuration. For other applications, the dimensions and configuration of grooves 104 may be varied to accommodate bone pins having different dimensions and configurations (not expressly shown). Bolt 106 or another suitable mechanical fastener may be used to engage first jaw and second jaw with each other to trap pins 22 or 26 therebetween.

First portion 40 preferably includes housing 43 having a generally elongated rectangular configuration. First drive screw 41 is preferably disposed within housing 43. Second portion 60 also includes elongated housing 63 and second drive screw 62. For the embodiment represented by external fixation device 30, housing 43 and 63 may have substantially the same general configuration and design. However, fixation devices may be formed in accordance with teachings of the present with housings having substantially different configurations and dimensions.

The length of housing 63 and second drive screw 62 may be substantially longer than the corresponding length of housing 43 and first drive screw 41. For other applications, housings 43 and 63 and drive screws 41 and 62 may have approximately the same length. For treatment of contracture at wrist 20, the length of housing 43 and first drive screw 41 may be selected based in part on the length of a patient's second metacarpal. The length of housing 63 and second drive screw 62 may be selected based in part on the length of a patient's radius.

For some applications housings 43 and 63 may be formed with different colors such as anodized black and anodized blue. The difference in color may be used to quickly identify housings with different dimensions or different axis of rotation. Also, labels such as A or B, distal or proximal, radial/ulnar or palmer/dorsal may be placed on respective housings 43 and 63 to aid with proper installation of an associated fixator.

Housing 43 preferably includes an elongated slot or opening 44. First drive screw 41 may be rotatably disposed within elongated slot 44. Threads 46 are preferably formed on the exterior of first drive screw 44 and engaged with a portion of first jaw 101 of clamp assembly 100a whereby rotation of first drive screw 41 will result in longitudinal movement of clamp assembly 100a relative to coupling assembly 80 and second portion 60.

Housing 63 preferably includes an elongated slot or opening 64. A plurality of threads 66 are preferably formed on the exterior of second drive screw 62 and engaged with a portion of first jaw 101 of clamp assembly 100b whereby rotation of second drive screw 62 will result in longitudinal movement of clamp assembly 100b relative to coupling assembly 80 and first portion 40. First drive screw 41 and second drive screw 62 may be used to incrementally and independently adjust longitudinal spacing between respective clamp assemblies 100a and 100b and coupling assembly 80.

First portion 40 and second portion 60 include respective first ends 51 and 71 and respective second ends 52 and 72. Coupling assembly 80 rotatably connects second end 52 of first portion 40 with second end 72 of second portion 60. For one embodiment of the present invention, coupling assembly 80 preferably allows controlled, incremental rotation of first portion 40 in a generally horizontal plane relative to second portion 60 which also corresponds generally with a plane extending through bone pins 22. Coupling assembly 80 also preferably allows controlled, incremental rotation of second portion 60 in a generally vertical plane relative to first portion 40 which also corresponds generally with movement perpendicular to the plane extending through bone pins 22. Controlled, incremental rotation of first portion 40 and second portion 60 may also be conducted independent of each other.

Coupling assembly 80 may include one or more gears to provide desired controlled, incremental rotation or articulation of first portion 40 and second portion 60 relative to each other. Various types of gear mechanisms and controls may be used to form coupling assembly 80 in accordance with teachings of the present invention. For some applications coupling assembly 80 may sometimes be referred to as a “geared joint” or as an “articulated coupling.”

For the embodiment of the present invention as shown in FIGS. 1-5 portions of coupling assembly 80 may be attached with or mounted on second end 52 of first portion 40. Corresponding portions of coupling assembly 80 may also be attached with or mounted on second end 72 of second portion 60. For purposes of describing the present invention, various components and features of coupling assembly 80 which are attached with or mounted on second end 52 of elongated portion 40 will be designated with “a.” Various components and features of coupling assembly 80 attached with or mounted on second end 72 of second portion 60 will be designated with “b”.

Coupling subassembly 80a disposed on second end 52 preferably includes worm gear 82a and worm screw or worm shaft 84a. Coupling subassembly 80b disposed on second end 72 preferably includes worm gear 82b and worm screw or worm shaft 84b. Worm gear 82a is preferably coupled with and rotatably secured to worm shaft 84a. For the embodiment of the present invention as shown in FIGS. 1-5, worm shaft 84a may be slidably disposed within opening or passageway 86 formed in end 52 of first portion 40. Retainers 87a and 88a may be secured with opposite ends of worm shaft 84a to rotatably maintain worm shaft 84a within opening 86a. For some applications laser welding techniques may be used to secure retainers 87a and 88a with worm shaft 84a when disposed within opening 86a. For one application retainers 87a and 88a contain an opening sized to accommodate the respective ends of worm shaft 84a in a press fit condition. End 89a of worm shaft 84a may be sized to be engaged with a “D” shaped socket. For other applications end 89a of worm shaft 84 a may be modified to have a generally hex shaped socket (not expressly shown). Also, end 89a of worm shaft 84a may be modified to accommodate rotation by hand (not expressly shown). Worm shaft 84b may have the same configuration and design as worm shaft 84a.

For the embodiment of the present invention as represented by external fixation device 30, end 52a of housing 43 preferably includes slot 54a sized to rotatably receive worm gear 82a therein. Opening or passageway 86a is also preferably formed in end 52. Passageway 86a and passageway 56a are oriented at approximately ninety degrees (90°) relative to each other. Clamp screw 90a is preferably inserted through passageway 56a and opening 83a in worm gear 82a. Clamp screw 90 and opening 83a are sized to allow rotation of worm gear 82a relative to clamp screw 90a. The end of clamp screw 90a is preferably engaged with keyed nut or captured nut 92a. Keyed nut or captured nut 92a is prevented from rotation relative to end 52. In addition to allowing rotation of worm gear 82a within slot 84a, clamp screw 90a may be rotated to compress or engage worm gear 82a in a desired location or lock worm gear 82a in a desired location.

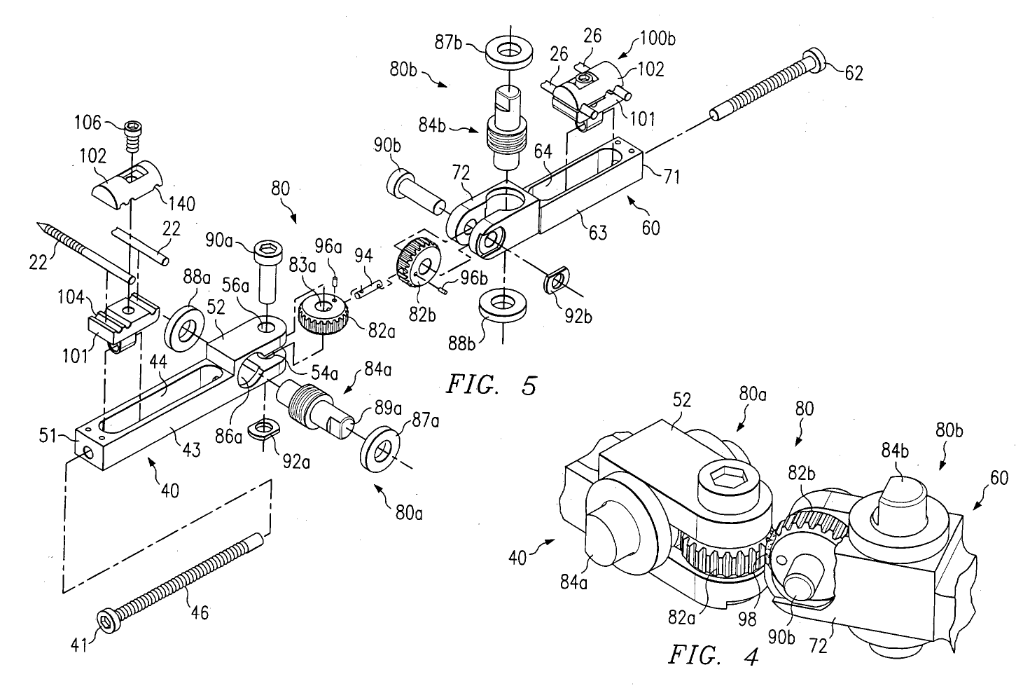

Worm gear 82a is preferably secured with and attached to worm gear 82b by connecting dowel 94 and cross pins 96b and 96a. See FIG. 5. In addition to connecting dowel 94, weld 98 may also be used to securely couple worm gear 82b with worm gear 82a. See FIG. 4.

Rotation of worm screw 84a will result in generally horizontal rotation or articulation of first portion 40 relative to second portion 60. In a similar manner rotation of worm screw 84b will result in generally vertical rotation or articulation of first portion 40 relative to second portion 60. When first portion 40 is has been placed in a desired location relative to second portion 60, clamp screw 90a may be used to securely lock worm gear 82a in its desired position. In a similar manner, after second portion 60 has been placed in its desired position, clamp screw 90b may be used to lock second portion 60a in its desired position relative to first portion 40.

During treatment of a contracture or other orthopedic indication, worm shafts 84a and 84b may be incrementally adjusted to independently vary the articulation and/or angulation of first portion 40 and second portion 60 relative to each other as appropriate for the desired treatment. During the treatment process first drive screw 41 and second drive screw 61 may also be adjusted to incrementally and independently modify the longitudinal position of respective clamps assemblies 100a and 100b relative to coupling assembly 80. Thus, external fixation device 30 provides at least six degrees of freedom or movement which may be incrementally and independently adjusted as required to treat the associated contracture or other orthopedic condition.

During treatment of a contracture or other orthopedic indication a small wire (sometimes referred to as a K wire) may be inserted into the associated bone or joint to provide a reference point for attachment of a fixation device incorporating teachings of the present invention. For the embodiment of the present invention as shown in FIG. 1, a small passageway (not expressly shown) may be formed in and extending longitudinally through clamp screw 90a. A K wire (not expressly shown) may be inserted at the center location of wrist 20. External fixation device 30 may then be inserted over the K wire and clamp assembly 100a engaged with bone pins 22 and clamp assembly 126 engaged with bone pins 26. One or more surgical releases or “Z” cuts may be formed in the soft tissue (tendon, ligament or muscle) associated with causing the contracture. During the healing process from the soft tissue release worm shafts 84a and 84b may be rotated as desired to return wrist 20 to its normal position. For some types of contractures a typical treatment process might include rotation of worm shafts 84a and 84b by approximately one half of a rotation per day for a period of ten to fifteen days. Also, drive screws 41 and 61 may be used to compensate and maintain desired longitudinal position of coupling assembly 80 relative to the K wire.

Although the present invention and its advantages have been described in detail, it should be understood that various changes, substitutions and alternations can be made herein without departing from the spirit and scope of the invention as defined by the following claims.

What is claimed is:

1. A fixation device for treatment of an orthopedic indication comprising:
a first portion and a second portion;
a first clamp assembly for releasably attaching the first portion with at least one bone pin;
a second clamp assembly for releasably attaching the second portion with at least one bone pin;
a coupling assembly joining the first portion with the second portion;
the coupling assembly operable to allow rotation of the first portion and the second portion relative to each other in generally vertical and horizontal planes; and
respective controls for incremental rotation in the horizontal plane independent of rotation in the vertical plane and incremental rotation in the vertical plane independent of rotation in the horizontal plane.

2. The device of claim 1 wherein the articulated coupling assembly further comprises two worm gear assemblies.

3. The device of claim 1 further comprising an external fixator.

4. The device of claim 1 further comprising the first portion having a length less than the length of the second portion.

5. The device of claim 1 further comprising the first portion and the second portion having respective elongated configurations defined in part by respective generally rectangular cross sections.

6. The device of claim 1 further comprising the first portion and the second portion having substantially the same length.

7. The device of claim 1 further comprising:
a first control for incremental positioning of the first clamp assembly relative to the coupling assembly independent of the position of the second clamp assembly; and
a second control for incremental positioning of the second clamp assembly relative to the coupling assembly independent of the first clamp assembly.

8. The device of claim 1 wherein the coupling assembly further comprises:
a first worm gear assembly having a first worm gear mounted on one end of the first portion;
a second worm gear assembly having a second worm gear mounted on an adjacent end of the second portion; and
the first worm gear assembly and the second worm gear assembly engaged with each other.

9. The device of claim 8 further comprising:
a first worm shaft operable to incrementally position the first worm gear; and
a second worm shaft operable to incrementally position the second worm gear.

10. The device of claim 8 further comprising:
a first clamp screw operable to lock the first worm gear in a desired position relative to the first portion; and
a second clamp screw operable to lock the second worm gear in a desired position relative to the second portion.

11. A method for treatment of an orthopedic indication comprising:
releasably attaching a first portion of a fixation device with at least a first bone pin;
releasably attaching a second portion of the fixation device with at least a second bone pin spaced from the first bone pin;
incrementally adjusting articulation of the first portion relative to the second portion and independent of the second portion; and
incrementally adjusting articulation of the second portion relative to the first portion and independent of the first portion.

12. The method of claim 11 wherein adjusting articulation between the first portion and the second portion of the fixation device further comprises adjusting a first worm gear and a second worm gear.

13. The method of claim 11 further comprising:
releasably attaching the first portion with the at least first bone pin by a first clamp assembly;
releasably attaching the second portion with the at least second bone pin by a second clamp assembly;
incrementally adjusting the position of the first clamp assembly relative to the second clamp assembly; and
incrementally adjusting the position of the second clamp assembly relative to the first clamp assembly.

14. The method of claim 11 further comprising:
incrementally adjusting articulation of the first portion relative to the second portion by rotation of a first worm gear; and
incrementally adjusting articulation of the second portion relative to the first portion by adjusting the position of a second worm gear.

15. The method of claim 11 further comprising:
securing the first portion in an articulated position relative to the second portion by clamping a first gear in a desired position; and
securing the second portion in a desired articulated position relative to the first portion by clamping a second worm gear in a desired position.

16. An external fixation device comprising:
a first portion and a second portion coupled with each other by a first worm gear assembly and a second worm gear assembly;
the first worm gear assembly operable to incrementally adjust rotation of the first portion relative to the second portion in a generally horizontal plane; and
the second worm gear assembly operable to incrementally adjust rotation of the second portion relative to the first portion in a generally vertical plane.

17. The external fixation device of claim 16 further comprising:
the first worm gear assembly having a first worm shaft and a first worm gear;
the second worm gear assembly having a second worm shaft and a second worm gear; and
the first worm gear securely attached with the second worm gear.

18. The external fixation device of claim 16 further comprising:
a first clamp assembly for releasably attaching the first portion with a pair of bone pins; and
a second clamp assembly for releasably attaching the second portion with at least a pair of bone pins.

19. The external fixation device of claim 16 further comprising:
a first control operable to adjust the rotation of the first portion relative to the second portion independent of the second gear assembly; and
a second control operable to adjust rotation of the second portion independent of the first worm gear assembly.
